(19) United States
(12) Patent Application Publication
(10) Pub. No.: US 2017/0340711 A1
(43) Pub. Date: Nov. 30, 2017

(54) Articular Cartilage Mimetics

(71) Applicant: New Jersey Institute of Technology, Newark, NJ (US)

(72) Inventors: Tamilvizhi Muthalagu, East Newark, NJ (US); George Collins, Maplewood, NJ (US); Treena L. Arinzeh, West Orange, NJ (US)

(73) Assignee: New Jersey Institute of Technology, Newark, NJ (US)

(21) Appl. No.: 15/674,160

(22) Filed: Aug. 10, 2017

Related U.S. Application Data
(63) Continuation of application No. 13/866,404, filed on Apr. 19, 2013.
(60) Provisional application No. 61/635,725, filed on Apr. 19, 2012.

(51) Int. Cl. A61K 38/39; A61L 27/48; A61L 27/52

(52) U.S. Cl. CPC A61K 38/39; A61L 27/52; A61L 27/48; A61L 2430/06

(57) ABSTRACT

A scaffold for promoting cartilage formation is provided that includes a crosslinked electrospun fiber, wherein the crosslinked electrospun fiber consists essentially of crosslinked gelatin. The crosslinked electrospun fiber is generally crosslinked with a crosslinker, and the crosslinker may be diisosorbide bisepoxide. The crosslinked electrospun fiber may be crosslinked by adding a crosslinker to a solution of gelatin at a desired concentration. The electrospun fiber may advantageously remain intact for 18 days or longer upon being immersed in an aqueous solution. A composition for promoting cartilage formation is also provided that includes the disclosed scaffold and a mesenchymal stem cell (MSC). The disclosed scaffold may include a crosslinked electrospun fiber that includes gelatin and sodium cellulose sulfate (NaCS), e.g., in an amount of up to 5% by weight of the amount of gelatin. A method for promoting cartilage formation is also provided that includes administering to a subject in need thereof a disclosed composition for promoting cartilage formation in the subject.

### CROSS REFERENCE TO RELATED PATENTS

The present application claims priority benefit to US Non-Provisional Patent Application No. 13/866,404, filed Apr. 19, 2013, and US Provisional Application No. 61/635,725, filed Apr. 19, 2012.

### FIELD OF THE INVENTION

This invention relates to articular cartilage mimetics and processes to make them.

### BACKGROUND OF THE INVENTION

Articular cartilage is a specialized type of tissue, lining the articulating surface of bone. It is a tissue with high load bearing, high wear resistance and low friction capacity. Articular cartilage is critical for movement of bones. It facilitates load support, and load transfer while allowing for rotational and translational movements of the bones. The statement that articular cartilage is crucial for daily activities is an understatement of it is importance in mobility of human beings. An injury or defect in articular cartilage drastically affects the activity of a person. The Centers for Disease Control and Prevention estimates that arthritis (a joint disorder caused due to cartilage loss) costs in excess of $128 billion per year and continues to be the most common cause of disability.

Most common reasons for articular cartilage damage include trauma and degenerative disease like arthritis. Unfortunately, the avascular, aneural and alymphatic nature of articular cartilage impede body's natural ability to repair and regenerate. Current clinical treatment for articular cartilage damage includes Autologous Chondrocyte Implantation (ACI), microfracture, autograft and allograft. All of these treatments are limited in their ability to regenerate functional cartilage in terms of composition and mechanics. Due to these limitations, there has been constant research promoting articular cartilage regeneration.

Articular cartilage is a fiber reinforced hydrogel composite of collagen fibers and proteoglycan-water gel, which is sulfated by glycosaminoglycans (GAGs). Regenerating this highly specific composition of articular cartilage is a critical challenge. Field of tissue engineering offers promising solutions, in which regeneration of articular cartilage is pursued through combinations of cells (e.g., chondrocytes or stem cells), and scaffolds (e.g., hydrogels, sponges, nanofibers, meshes) to guide tissue formation. Despite these advances, there has not yet been a process developed to mimic articular cartilage with the subtle variations in composition and mechanical properties as observed in native articular cartilage.

Recent studies have attempted to imitate the spatially varying mechanical properties of cartilage using combinations of synthetic and natural polymer hydrogels. Biomaterial scaffolds made from natural polymers gain importance due to their similarity to natural tissue compositions. Fibrous and hydrogel scaffolds from natural polymers are extensively used in tissue engineering because of their ability to mimic the ECM architecture. Though studies have separately fabricated natural polymers into fiber and hydrogel, there was no attempt to combine the components into a fiber reinforced hydrogel as a scaffolding material.

### SUMMARY OF THE INVENTION

It has now been found that certain composites of electrospun fibers and hydrogels are articular cartilage mimetics to closest proximity known.

More particularly, in one embodiment of the invention, the composite is composed made from gelatin/sodium cellulose sulfate blends to generate fiber reinforced hydrogel composite.

In another embodiment the composite and the hydrogel are composed of the same materials.

More particularly, the hydrogel and the electrospun fiber are both composed of gelatin and codium cellulose sulfate.

In a particular embodiment, the hydrogel and the electrospun fiber are both crosslinked.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. So that those having ordinary skill in the art will have a better understanding of how to make and use the disclosed gel composites, reference is made to the accompanying figures wherein:

FIG. 1 shows schematic comparison of native articular cartilage and fiber reinforced hydrogel composite developed that mimic the articular cartilage;

FIG. 2 shows the compressive modulus of hydrogels;

FIG. 3 shows the shear modulus of ‘initial hydrogels’;

FIG. 4 shows the percentage of weight increase in hydrogels;

FIG. 5 shows SEM images of lyophilized hydrogels;

FIG. 6 shows SEM image of fibers made with PBS/0% NaCS;

FIG. 7 shows SEM image of fibers made with PBS/5% NaCS;

FIG. 8 shows microscope images of day 2 and day 5 images of fibroblasts on hydrogel disks made with PBS;

FIG. 9 shows day 1 confocal images of hMSCs on fiber and composite disks; and

FIG. 10 shows day 4 and 7 confocal images of hMSCs on fiber and composite disks.

### DETAILED DESCRIPTION OF THE INVENTION

This invention relates to fiber reinforced hydrogel composite similar to articular cartilage. One embodiment of the invention was fabricated using gelatin and sodium cellulose sulfate.

Gelatin is a natural polymer obtained by denaturation of collagen. Sodium cellulose sulfate is a natural polymer derived from cellulose, with structural similarity to glycosaminoglycan in proteoglycan. In one embodiment of the invention, gelatin and sodium cellulose sulfate is used as principal compounds in fabricating both fibrous and hydrogel components of fiber reinforced hydrogel composite. The fibers were fabricated using a technique called electrospinning whereas hydrogels were solution casted.

Before arriving at the point of generating fiber reinforced composite, the individual fiber and hydrogel components where evaluated for stability, mechanical properties and cell culture studies to assess their suitability in regenerating articular cartilage. In addition specific representative embodiments of the composite were evaluated in cell studies.

Specific and unique combinations of gelatin and sodium cellulose sulfate were fabricated in to fibers and hydrogels. Hydrogels were made using water and PBS (Phosphate Buffer saline) as solvents. Fibers were made using only PBS as solvent. Hydrogels were assessed for swelling ratio, surface morphology, stability and mechanical properties like compressive modulus, and shear modulus. Fibers were assessed for stability and surface morphology. Fiber reinforced hydrogels were fabricated using suction. Fibers, hydrogels and composites were added with crosslinker diisosorbide epoxide to increase stability. Also, fibers, hydrogels and composites were cultured with hMSCs (human Mesenchymal Stem Cells) and fibroblasts.

A unique element of the exemplary fabrication of fiber reinforced composite material is sulfation of the composite. Both the fiber component and hydrogel component were sulfated. Articular cartilage proteoglycan-water gel is sulfated due to the presence of sulfate groups in GAGs. Also, the collagen fibers in articular cartilage are capable of attaching to small proteoglycans like decorin, making the collagen element of the articular cartilage sulfated.

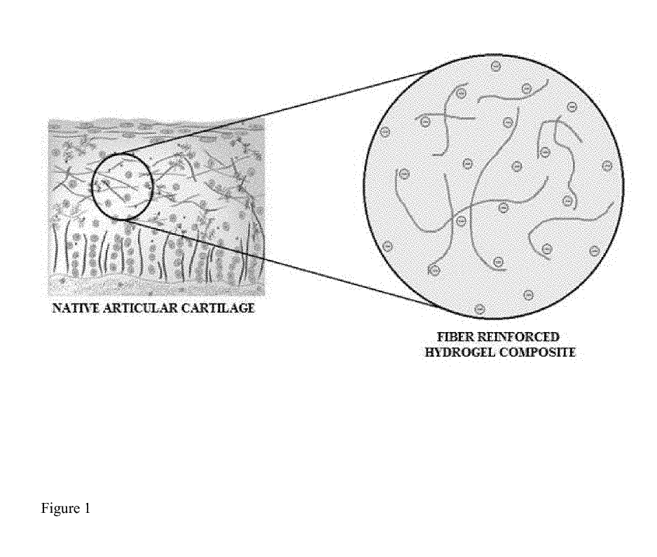

FIG. 1 shows a schematic comparison of native articular cartilage and a fiber reinforced hydrogel composite of the invention that mimics the articular cartilage.

The fibrous network was studied by electro spinning gelatin/NaCS blends because in articular cartilage, GAGs are attached to a protein core via tetrasaccharide link Stability of fibers with different percentages of crosslinker was assessed by immersing fibers in water and in PBS. Irrespective of percentage of NaCS used in making the fiber scaffolds, the scaffolds with 5 and 10 percentage of crosslinker dissolved after 4 days both in water and PBS. Only the fibers made with 20% crosslinker remained intact without dissolving for more than 18 days.

Compressive modulus data of hydrogel dictates that with addition of crosslinker, the compressive modulus appears to decrease. Experiments to assess the shear modulus of hydrogels imply that with increase in percentage of crosslinker the shear modulus increases with up to 5% of crosslinker. But shear modulus decreases when the added crosslinker is present at more than 5%. The ratio of swelling of hydrogels increases with increase in percentage of NaCS except when hydrogels were made with PBS and swollen in PBS. Addition of crosslinker to hydrogels decreases extent of swelling for hydrogels made with PBS but increases for hydrogels made with water. It appeared that 5% of crosslinker was sufficient to control swelling.

Cell study was also performed on fibers, hydrogel disks and fiber reinforced hydrogel composites. Irrespective of the type of cells, fiber scaffolds exhibited good cell attachment and growth. Cells grow on fiber exhibit stretching along the length of the fibers but in hydrogels/composites the cytoskeleton seems to have stretching in all directions.

Various hydrogels were prepared as described below. A list of the hydrogels prepared is shown in Table 1.

TABLE 1
Solvent

| | |
|---|---|
| PBS | Water |

| | | | |
|---|---|---|---|
| 0% NaCS | 0% CL | 0% NaCS | 0% CL |
| | | | 3% CL |
| | 5% CL | | 5% CL |
| | 10% CL | | 10% CL |
| | 20% CL | | 20% CL |
| 5% NaCS | 0% CL | 5% NaCS | 0% CL |
| | | | 3% CL |
| | 5% CL | | 5% CL |
| | 10% CL | | 10% CL |
| | 20% CL | | 20% CL |
| 10% NaCS | 0% CL | 10% NaCS | 0% CL |
| | 5% CL | | 5% CL |
| | 10% CL | | 10% CL |
| | 20% CL | | 20% CL |
| 20% NaCS | 0% CL | 20% NaCS | 0% CL |
| | 5% CL | | 5% CL |
| | 10% CL | | 10% CL |
| | 20% CL | | 20% CL |

Compressive modulus of hydrogels was determined using DMTA. Compressive modulus was calculated from the initial slope of stress verses strain curve. It is a measure of the capability of a material to withstand axially directed pushing forces. Compressive modulus was measured for hydrogels made with gelatin solutions containing different percentages of NaCS (0, 5, 10, and 20%) and different percentages of crosslinker (0, 5, 10, and 20%), using water and PBS as solvents. Compressive modulus of hydrogels swollen in water and PBS was also measured. The terms “initial hydrogels”, “swollen in water” and “swollen in PBS” represents the hydrogels that were tested before swelling, tested after swelling in water, tested after swelling in PBS.

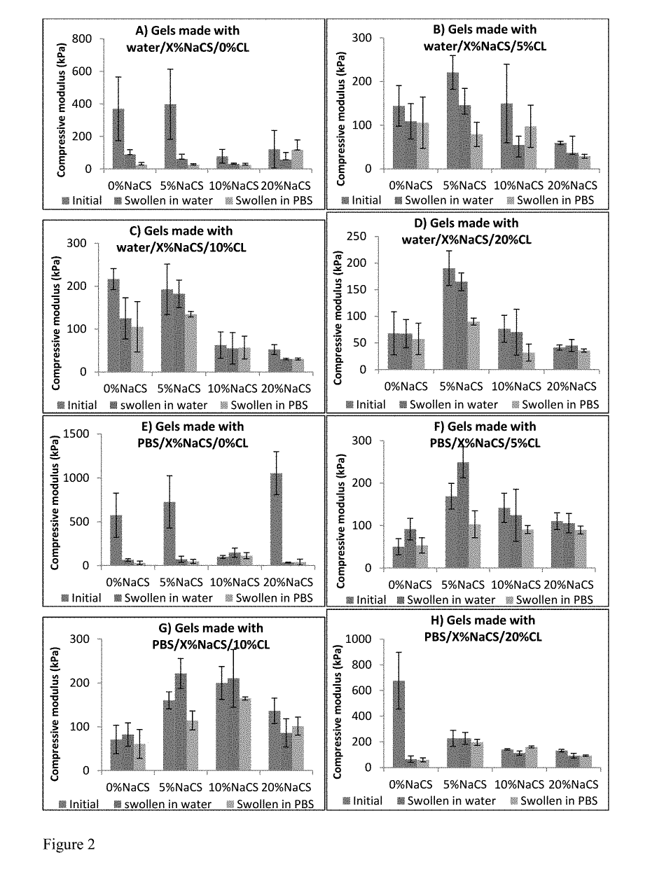

FIG. 2 shows the compressive modulus data for initial hydrogels, hydrogels swollen in water, hydrogels swollen in PBS that were made with both water and PBS as solvents. When the gels with crosslinker were stretched manually they were more elastic when compared to the gels without crosslinker. Hence, the hydrogels were also evaluated for shear strength.

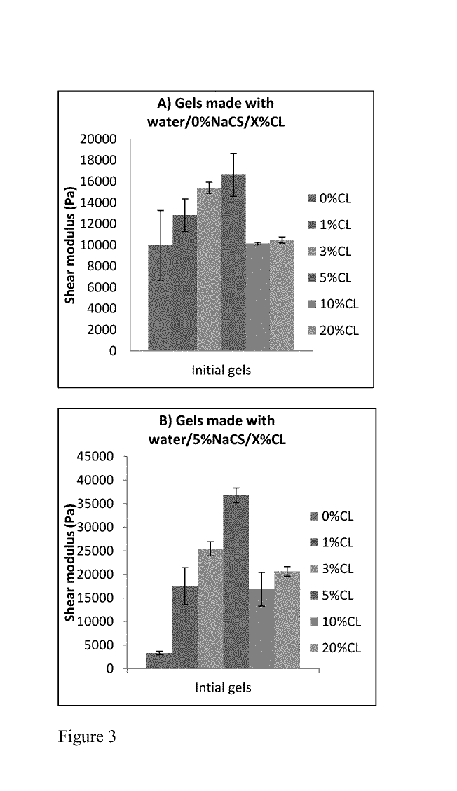

Shear modulus of hydrogels was determined using RMS-800. Shear modulus measures the material's response to shearing strains. It is concerned with deformation of solid when force is applied parallel to one surface while it is opposite surface is held fixed. Shear tests were performed on hydrogels made with water with different percentages of NaCS (0, 5%) and different percentages of crosslinker (0, 1, 3, 5, 10, and 20%). The term “initial hydrogels” represents the hydrogels that were tested before swelling. The results are shown in FIG. 3.

In FIG. 3A, the shear tests on hydrogels reflect an increase in shear modulus with addition of crosslinker up to 5% but decreases with very high concentrations of crosslinkers (10 and 20%). This trend of shear modulus to increase up to 5% of crosslinker and decrease with addition of 10 and 20% of crosslinker was reproducible in other hydrogel combination shown in FIG. 3B.

Swelling of hydrogels was evaluated by measuring the weight increase after rehydrating from the lyophilized state by swelling in same initial volume (5 ml) of water and PBS for 16-20 hours. Swelling was measured for hydrogels made with gelatin solutions containing different percentages of NaCS (0, 5, 10, 20%) and different percentages of crosslinker (0, 5, 10, 20%). Hydrogels were made with water and in PBS.

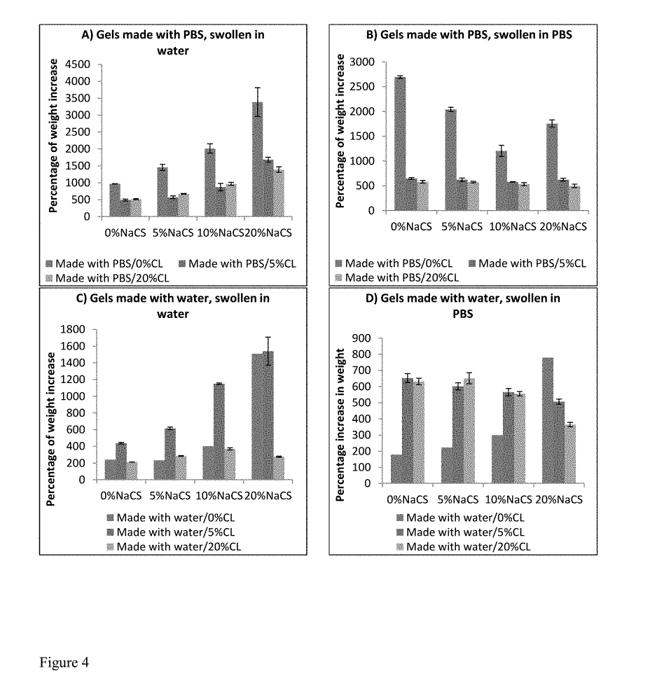

The hydrogels exhibit different swelling ratio on each case, as shown in FIG. 4. It is found that swelling of hydrogels was dependent on concentration of NaCS, addition of crosslinker and Donnan osmotic equilibrium. Overall, the hydrogels without crosslinker exhibited an increase in swelling with increase in concentration of NaCS as shown in FIGS. 4A, 4B, and 4C, except for the hydrogels made with PBS and swollen in PBS (FIG. 4D) which showed a decrease in swelling with increase in concentration of NaCS. For the hydrogels with crosslinker there is an increase/decrease in swelling depending on the aqueous environment they were swollen in. Hydrogels with crosslinker swollen in PBS exhibited decrease in swelling (FIG. 4A and FIG. 4B), whereas the hydrogels with crosslinker swollen in water exhibited an increase in swelling (FIG. 4C and FIG. 4D). There is no significant difference in swelling with increase in crosslinker percentage from 5 to 20%.

Stability of hydrogels was evaluated by immersing hydrogels in water and in PBS. Hydrogels were considered stable until it starts to dissolution. Table 2 compares the stability of hydrogels that were not dried verses the hydrogels that were dried for 36 hours. The hydrogels evaluated for stability without drying were made with PBS/0, 5, 10 and 20% of NaCS/20% CL, whereas the hydrogels evaluated for stability after drying for 36 hour were made with water/0, 5, 10 and 20% of NaCS/5% CL.

TABLE 2
Stability of Hydrogels

| Hydrogels | Without drying | Air dried for 36 hours |
|---|---|---|
| Swollen in water | Dissolved after day 3 | Dissolved after day 7 |
| Swollen in PBS | Dissolved after day 3 | Dissolved after day 7 |

Fibers produced by electro spinning were assessed for stability. Fibers were considered to be stable before initiation of dissolution. Table 3 shows the stability of fibers made with PBS/without NaCS (0% NaCS)/5, 10 and 20% CL. The fibers were immersed in water and in PBS. The data suggests that only fiber with 20% CL were stable without dissolution for longer period of 18 days than any other crosslinker percentage. Likewise, stability data of fibers made with PBS/5% NaCS/5, 10 and 20% CL Table 4 also suggests that only fibers with 20% CL had longer stability of 18 days than any other crosslinker percentage.

TABLE 3
Stability of Fibers Made with PBS/0% NaCS

| Fibers made with | | | |
|---|---|---|---|
| PBS/0% NaCS | 5% CL | 10% CL | 20% CL |
| Swollen in water | Dissolved | Dissolved | Remained intact for more |
| | after day 6 | after day 6 | than 18 days |
| Swollen in PBS | Dissolved | Dissolved | Dissolved after day 4 |
| | after day 2 | after day 3 | |

TABLE 4
Stability of Fibers Made with PBS/5% NaCS

| Fibers made with | | | |
|---|---|---|---|
| PBS/5% NaCS | 5% CL | 10% CL | 20% CL |
| Swollen in water | Dissolved | Dissolved | Remained intact for |
| | after day 3 | after day 6 | more than 18 days |
| Swollen in PBS | Dissolved | Dissolved | Dissolved after day 4 |
| | after day 2 | after day 3 | |

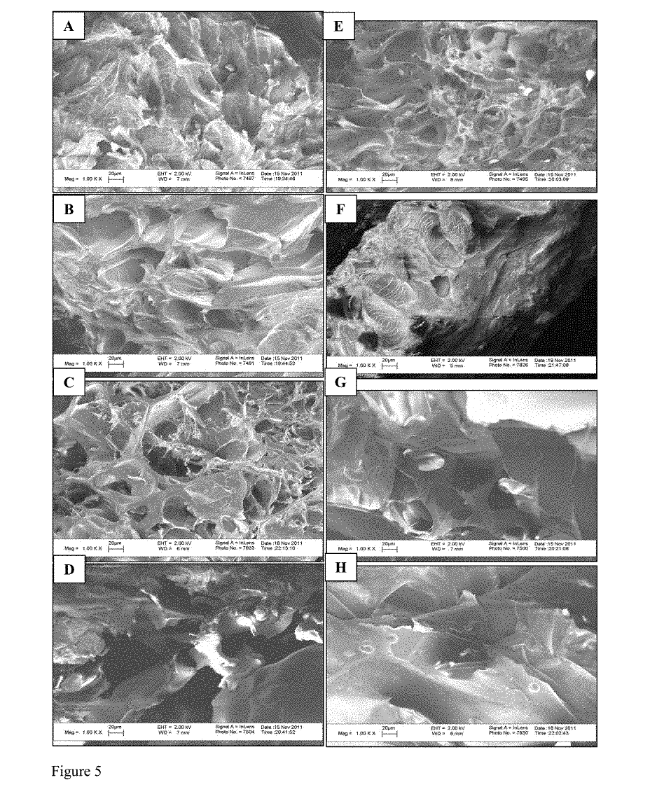

The porosity of hydrogels was examined using SEM. FIG. 5 shows the pores present in the initial freeze dried hydrogels and freeze dried hydrogels swollen in water. Freeze dried swollen hydrogels (FIGS. 5E, 5F, 5G, 5H) appears to have larger pore size when compared to initial freeze dried hydrogels (FIGS. 5A, 5B, 5C, 5D). All the gels were made without adding crosslinker.

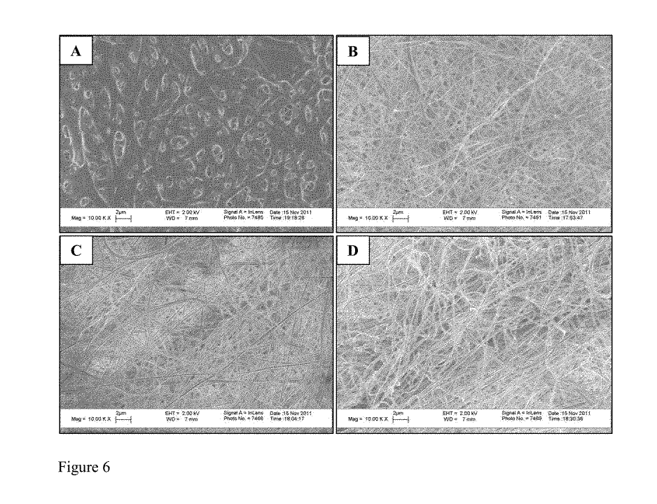

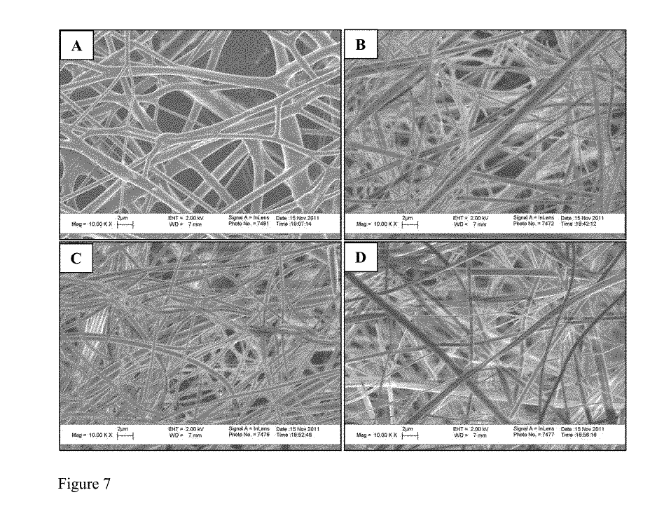

Fibers produced by electrospinning were examined for morphology using SEM (FIGS. 6 and 7). The fibers with crosslinker were post treated by heating at 121° C. for 4 hours to allow for crosslinking The fiber without crosslinker was also heated at 121° C. for 4 hours for comparison. Fibers appear to be coalescing for fibers without crosslinker (FIG. 6A). When comparing the fibers without crosslinker (FIG. 6A) to fibers with crosslinker (FIG. 6B, 6C, 6D), it appears as though with addition of crosslinker the fibers were distinct and more pronounced. Comparing FIGS. 6 and 7, it is understood that the fiber diameter increases with addition of NaCS.

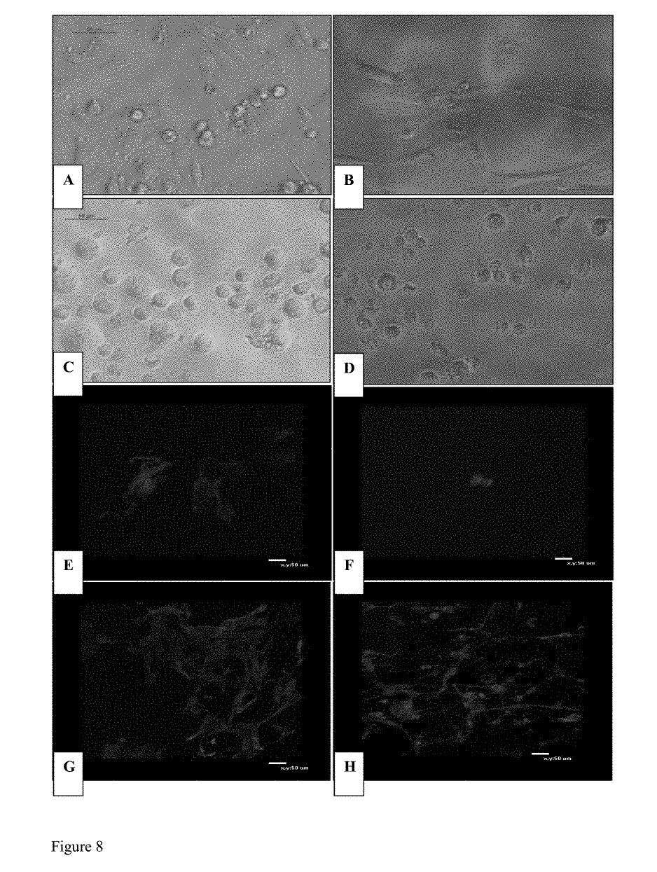

Adhesion of fibroblasts and hMSCs on the biomaterial scaffolds of fibers, hydrogel disks and composites was examined by Actin-DAPI staining. All scaffolding materials were made from gelatin solutions with 0% NaCS/20%crosslinker and 5% NaCS/20%crosslinker, using PBS as solvent. The distribution of actin microfilaments and nucleus was carefully observed. Multiple dishes were prepared for the experiments and were stained at time points one, four and seven days to examine whether the hMSCs adhere well to the scaffold system. Scaffolds seeded with fibroblasts were stained at time points 2 and 5 days. Scaffolds seeded with hMSCs were stained at time points 1, 4 and 7 days. FIG. 8 shows microscope images of day 2 and day 5 images of fibroblasts on hydrogel disks made with PBS.

FIGS. 8A and 8B are phase contrast microscope images of fibroblasts seeded on hydrogel scaffolds made with PBS/without NaCS (0% NaCS)/20% CL on day 2 and day 5. FIGS. 8C and 8D are hydrogel scaffolds made with PBS/with 5% NaCS/20% CL on day 2 and day 5. FIGS. 8A and 8C were imaged when the cells were alive. FIGS. 8B and 8D were imaged after fixing of the cells. FIGS. 8E, 8F, 8G and 8H were confocal microscope images of fibroblasts taken on day 5 for fiber and hydrogel disks seeded with fibroblasts. FIGS. 8E and 8F are hydrogels without NaCS and with 5% NaCS. FIGS. 8G and 8H are fibers made with PBS/without NaCS (0% NaCS)/20% CL and fibers made with PBS/with 5% NaCS/20% CL. On both fibers with and without NaCS, the cells showed good attachment and stretching.

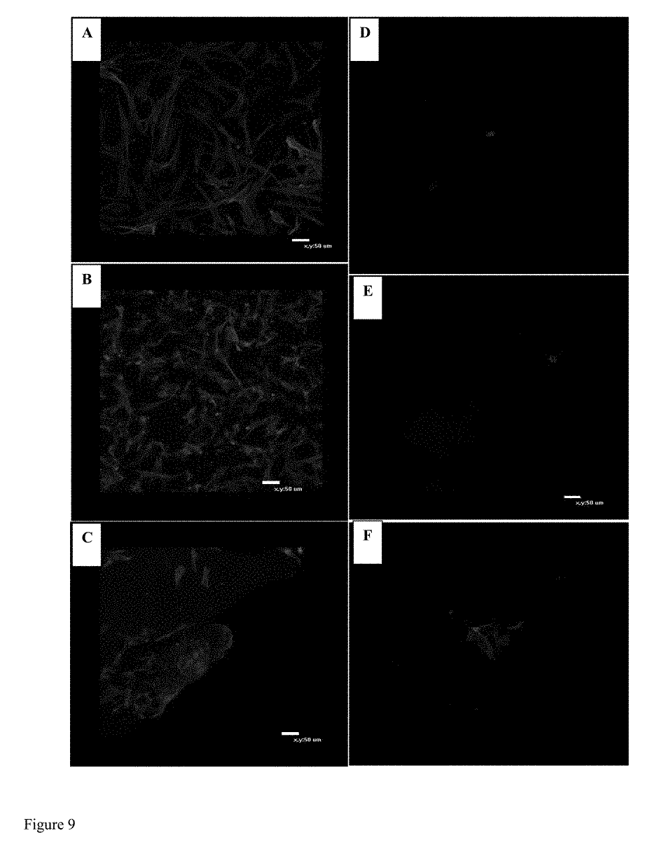

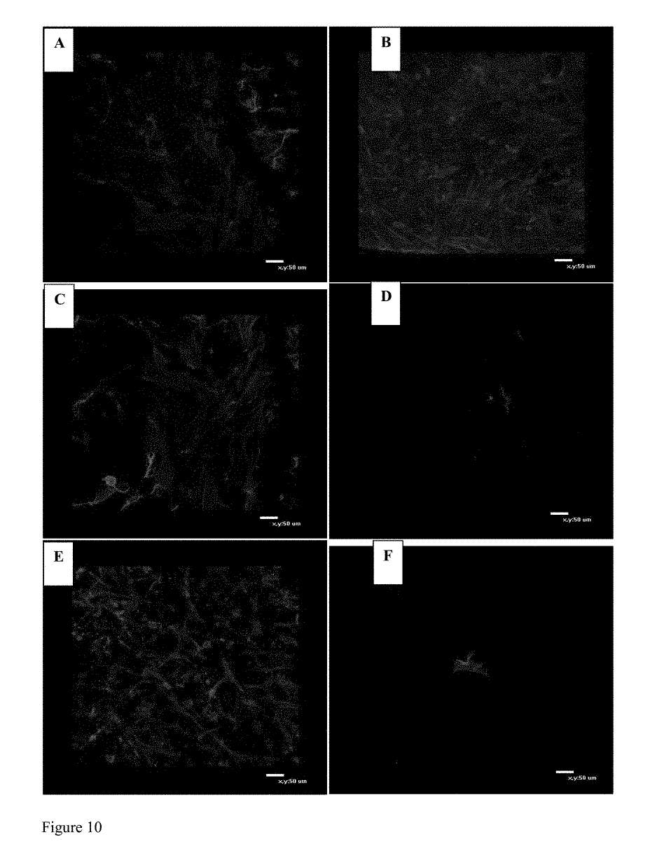

FIG. 9 shows 1 day 1 confocal images of hMSCs on fiber and composite disks where A) 0% NaCS fiber B) 5% NaCS fiber C) Aggregate formation in 5% NaCS fiber D) Fiber reinforced composite hydrogel with 5% NaCS fiber, 0% NaCS hydrogel E) Fiber reinforced composite hydrogel with 5% NaCS fiber, 5% NaCS hydrogel F) 5% NaCS hydrogel disks. FIG. 10 shows day 4 and 7 1 confocal images of hMSCs on fiber and composite disks, where Day 1 A)Fiber reinforced composite hydrogel with 5% NaCS fiber, 0% NaCS hydrogel B) Fiber reinforced composite hydrogel with 5% NaCS fiber, 5% NaCS hydrogel. Day 7 images, C) 0% NaCS fiber D) 5% NaCS fiber E) 0% NaCS hydrogel disk F) 5% NaCS hydrogel disk. FIGS. 9A and 9B are day 1 confocal microscope images of hMSCs seeded on fiber without NaCS (0% NaCS) and fiber with 5% NaCS. FIG. 9C shows the aggregate formation in fibers with 5% NaCS on day 1. FIG. 10C and 10D are day 7 images of hMSCs on fibers without NaCS (0% NaCS) and with 5% NaCS. Comparison of day 1 and day 7 images of hMSCs on fibers exhibits a significant increase cell number as seen visually.

Comparison of FIGS. 9F, 10E and 10F suggests that though hMSCs didn't show much of attachment on day 1 they were able to attach, stretch and grow on day 7. The cell attachment and growth of hMCS and fibroblasts were similar in a way that they were clearly able to sense the presence of NaCS in hydrogels.

In fiber reinforced hydrogel scaffolds (FIGS. 9D, 9E, 10A and 10B) there was no hMSCs was found attached on day 1 and day 7, but there was attachment in day 4.

### MATERIALS AND METHODS

### Materials

### Gelatin

Gelatin from bovine skin, type B was purchased from Sigma-Aldrich. Sodium cellulose sulfate (NaCS) was generously provided by Dextran Products Ltd., (Scarborough, Ontario, Canada). The molecular weight of sodium cellulose sulfate is 3.04×10⁶ g/mol. The sulfur content of sodium cellulose sulfate as reported by Dextran Products Ltd. is 18.2%. Each cellulose unit has at least two sulfate groups. The structure of NaCS with two sulfate groups per cellulose unit is shown in formula (1). The solvents water and PBS were purchased from Fisher Scientific. All materials were used as received without any further treatment.

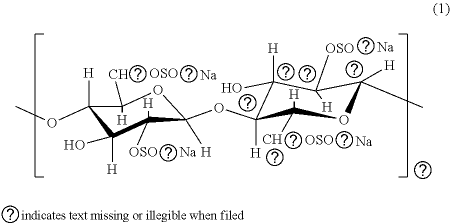

### Chemical Crosslinker

Diisosorbide bisepoxide (Dr. Wills B. Hammond, New Jersey Institute of Technology, Department of Biomedical Engineering, Newark, N.J./ Batch # 169/66, Date May 13, 2011) was the chemical crosslinker used in this study. The chemical structure of diisosorbide bisepoxide is shown as formula (2).

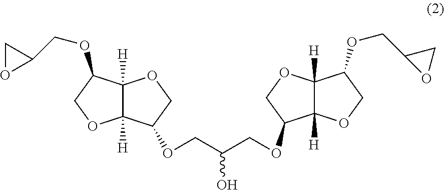 [SMILES: OC(CO[C@H]1CO[C@H]2[C@@H]1OC[C@H]2OCC1CO1)CO[C@H]1CO[C@H]2[C@@H]1OC[C@H]2OCC1CO1]

### Hydrogel Preparation

Deionized water and phosphate buffer saline were used as solvents in preparing hydrogels. Hydrogels were prepared using gelatin solutions with different NaCS concentrations were mixed well by stirring continuously for about 2 hours at 60° C. Solutions of 0%, 5%, 10% and 20% of NaCS (based on gelatin) in water or PBS with gelatin (24% w/w water or PBS) were used for all experiments. Blends of gelatin/NaCS were casted into disks in petri plates and allowed to gel at room temperature. For crosslinked hydrogel preparation, 5, 10 and 20% of crosslinker (based on solid weight of solution) was added after gelatin/NaCS dissolution and stirred for 10 minutes. After casting cylindrical samples of gels were cut out using biopsy punch (10 mm inner diameter, Acuderm Inc. USA,) for further experiments.

### Fiber Fabrication

Fibers were prepared using the technique called electro spinning Gelatin solutions for electro spinning were prepared by adding 0%, and 5% of NaCS (based on gelatin) to gelatin (24% w/w PBS) with PBS as solvent and stirring continuously for about 2 hours at 60° C. Crosslinking of fibers was done by adding various percentage (5, 10, 20% based on solid weight of solution) of crosslinker to well mixed solution of gelatin and NaCS blend, stirred for about 10-15 minutes and then electrospun. These electrospun fibers were then post treated by heating at 121° C. for 4 hours.

Electro spinning was carried out using electro spinning apparatus known in the art. The syringe (10 ml plastic syringe) contained the solution and was placed inside an insulated chamber maintained at a temperature of 60° C. to keep the solution viscosity low enough to be electrospun. A needle was attached as a spinneret to the syringe. The syringe was driven by a syringe pump (New Era pump systems Inc.). Compressed air was heated using inline heating coil which was then fed in to hot jacket surrounding syringe to maintain temperature of 60° C. The high voltage of 20-30 kV was applied using voltage power supply. The needle was of 12 gauge (inner diameter of 2.16 mm). The stainless steel collector plate was used to collect fibers and it was electrically grounded. The distance between the needle tip and collector plate was maintained between 20-25 cm. The flow rate on the syringe pump was set between 5-9 ml/hr.

### Lyophilization

The FreeZone plus 2.5 Liter cascade benchtop freeze dry systems' from Labconco Corporation was used for lyophilization. Swelling of hydrogels was assessed after lyophilization. The impact on swelling on adding crosslinker was also assessed. Hydrogels of NaCS/gelatin blends with and without crosslinker that was prepared, and lyophilized. The lyophilized samples were then swollen in water and PBS for 24 hours. The percentage of weight increase was calculated using the formula

$$Percentage of weight increase = \frac{W - {W}_{o}}{{W}_{o}} \times 100$$

where,

Wₒ is weight before swelling.

W is weight after swelling.

### Stability Studies

Stability studies were performed to assess ability of samples to be preserved without hydrolyzing when immersed in water and PBS. The crosslinked fibers and hydrogels were prepared and cut with biopsy punches. The cut samples were then immersed in water and PBS. The samples were considered stable until the initiation of dissolution.

### Material Characterization

### Compression Test

Dynamic Mechanical Thermal Analyzer (DMTA) was used for the compression test of hydrogels. Rheometric Scientific DMTA-IV is computer-controlled, having temperature range of −150° C. to 600° C. and displacement amplitudes from 0.5 to 128 microns.

The, DMTA was used to measure Young's modulus while applying uniform compressive force. Predefined compressive load of 1.0 g was applied on cylindrical samples (Approximate diameter of 10 mm and height of 2 mm) at a strain rate of −0.001/s for 60s. Young's modulus was measured from the initial slope of stress-strain curve.

### Compression Tests

### Shear Test

A Rheometric Mechanical Spectrometer (RMS-800) was used to measure shear modulus by applying dynamic strain sweep at a frequency of 6.28 radians using parallel plate geometries. Stain was applied in range from 1 to 100% using constant static force with a maximum displacement of 3 mm in a rate of 0.01 mm/s.

Scanning Electron Microscopy

LEO 1530VP SEM was used to study surface morphology of electrospun mats and freeze dried hydrogels. The samples were placed on the stub using double sided carbon tape. Samples were coated before placing in SEM vacuum chamber, using a sputter machine to produce thin layer of carbon on to the surface of electrospun mats and hydrogels.

### Composite Fabrication

Fiber reinforced hydrogel composites were fabricated by applying suction. Fibers fabricated by electro spinning and hydrogels solutions (solutions of gelatin/NaCS blends with crosslinker) were brought together under suction to fabricate composites. Composite fabrication was accomplished by placing fiber on a filter support and placing hydrogel solution over the fiber while suction was applied, thus forcing the hydrogel solution into the volume of electrospun mat.

### Cell Study

### Fibroblast Cell Culture

Fibroblasts were cultured in Dulbecco's modified eagle's medium (DMEM, Gibco) containing 4.5 g/L Glucose, L-Glutamine, and Sodium Pyruvate. DMEM was supplemented with 10% Fetal Bovine Serum (FBS, Gibco), 1% Pencillin/Streptomycin (P/S, Hyclone). Cells were cultured in fibrous scaffold, hydrogel disks scaffolds in 96 well tissue culture plate and kept in a humidified environment in 37° C/10% CO2. Cell culture medium was changed on Day 3 in 5 days study.

hMSCs Cell Culture

Human Mesenchymal Stem Cells(hMSCs) were cultured in basal growth media containing 10% Hyclone fetal bovine serum (FBS, Fisher Scientific), 1% Anti-Anti (Antibiotic-Antimycotic, Invitrogen) and Dulbecco's Modified Eagle Medium (DMEM, Invitrogen). Cells were cultured on the fibrous scaffolds, hydrogels disk scaffolds and fiber reinforced hydrogel scaffolds in 96 well tissue culture plate and kept in a humidified environment at 37° C./10% CO2 . Cell culture medium was changed on Day 3 in 7 days study.

### Actin-DAPI Staining

For immunofluorescence staining, double-stranded DNA of the cell nuclei was stained by 4, 6-diamidino-2-phenylindole dihydrochloride (DAPIJnvitrogen) and its cytoskeleton was stained by adding Rhodamine-Phalloidin (Invitrogen). Cells cultured scaffolds were gently washed with PBS to remove unattached cells. Paraformaldehyde 4% (Sigma-Adrich) solution in PBS was added in each well and incubated for 20 min at room temperature to fix the cells. After washing with PBS, 0.1% Triton X-100 (Sigma-Aldrich) in PBS was added for 5 minutes to permeabilize the fixed cells. Again after washing twice with PBS, Fluorescein-Phalloidin in PBS was added in each well and incubated for an hour at room temperature. After rinsing with PBS, cell nuclei were stained with DAPI and were visualized by confocal microscope (Nikon Instruments Inc.).

Although the systems and methods of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

1. A scaffold for promoting cartilage formation comprising a crosslinked electrospun fiber, wherein the crosslinked electrospun fiber consists essentially of crosslinked gelatin.

2. The scaffold of claim 1, wherein the crosslinked electrospun fiber has been crosslinked with a crosslinker, and wherein the crosslinker is diisosorbide bisepoxide.

3. The scaffold of claim 2, wherein the crosslinked electrospun fiber has been crosslinked by adding a crosslinker to a solution of gelatin to achieve a concentration of the crosslinker in the solution of about 5% to about 20% based on solid weight of the solution.

4. The scaffold of claim 3, wherein the electrospun fiber has been crosslinked by adding a crosslinker to a solution of gelatin to achieve a concentration of the crosslinker in the solution of about 5% based on solid weight of the solution.

5. The scaffold of claim 3, wherein the electrospun fiber has been crosslinked by adding a crosslinker to a solution of gelatin to achieve a concentration of the crosslinker in the solution of about 10% based on solid weight of the solution.

6. The scaffold of claim 3, wherein the electrospun fiber has been crosslinked by adding a crosslinker to a solution of gelatin to achieve a concentration of the crosslinker in the solution of about 20% based on solid weight of the solution.

7. The scaffold of claim 6, wherein the electrospun fiber remains intact for 18 days or longer upon being immersed in an aqueous solution.

8. A composition for promoting cartilage formation comprising the scaffold of claim 1 and a mesenchymal stem cell (MSC).

9. A scaffold for promoting cartilage formation comprising a crosslinked electrospun fiber, wherein the crosslinked electrospun fiber comprises gelatin and sodium cellulose sulfate (NaCS) in the amount of up to 5% by weight of the amount of gelatin.

10. The scaffold of claim 9, wherein the electrospun fiber has been crosslinked with a crosslinker, and wherein the crosslinker is diisosorbide bisepoxide.

11. The scaffold of claim 10, wherein the electrospun fiber has been crosslinked by adding a crosslinker to a solution of gelatin to achieve a concentration of the crosslinker in the solution of about 5% to about 20% based on solid weight of the solution.

12. The scaffold of claim 11, wherein the electrospun fiber has been crosslinked by adding a crosslinker to a solution of gelatin to achieve a concentration of the crosslinker in the solution of about 5% based on solid weight of the solution.

13. The scaffold of claim 11, wherein the electrospun fiber has been crosslinked by adding a crosslinker to a solution of gelatin to achieve a concentration of the crosslinker in the solution of about 10% based on solid weight of the solution.

14. The scaffold of claim 11, wherein the electrospun fiber has been crosslinked by adding a crosslinker to a solution of gelatin to achieve a concentration of the crosslinker in the solution of about 20% based on solid weight of the solution.

15. The scaffold of claim 14, wherein the electrospun fiber remains intact for 18 days or longer upon being immersed in an aqueous solution.

16. A composition for promoting cartilage formation comprising the scaffold of claim 9 and a mesenchymal stem cell (MSC).

17. A method for promoting cartilage formation, the method comprising administering to a subject in need thereof the composition of claim 8, thereby promoting cartilage formation in said subject.

18. A method for promoting cartilage formation, the method comprising administering to a subject in need thereof the composition of claim 16, thereby promoting cartilage formation in said subject.
